(19) United States
(12) Patent Application Publication
(10) Pub. No.: US 2012/0046457 A1
(43) Pub. Date: Feb. 23, 2012

(54) PREPARATION OF DECITABINE

(75) Inventors: Naveen Kumar Kolla, Hyderabad (IN); Uday Kumar Neelam, Hyderabad (IN); Sudhakar Reddy Baddam, Nalgonda (IN); Srinivas Gangula, Karimnagar (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/282,921

(22) Filed: Oct. 27, 2011

Related U.S. Application Data
(63) Continuation of application No. PCT/US2010/032352, filed on Apr. 26, 2010.
(60) Provisional application No. 61/256,562, filed on Oct. 30, 2009.
(60) Provisional application No. 61/311,541, filed on Mar. 8, 2010.

(30) Foreign Application Priority Data
Apr. 27, 2009 (IN) 971/CHE/2009
Sep. 30, 2009 (IN) 2372/CHE/2009

(51) Int. Cl. C07H 19/12; C07H 1/06

(52) U.S. Cl. 536/28.3

(57) ABSTRACT

The present application relates to processes for the preparation and purification of decitabine structurally represented by formula (I):

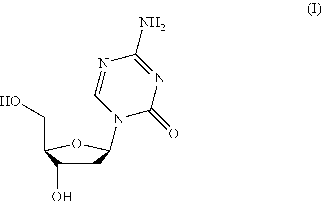

and to processes for the preparation of a crystalline form of decitabine.

The present application relates to processes for the preparation and purification of decitabine.

The drug compound having the adopted name “decitabine” has chemical names: 4-amino-1-(2-deoxy-β-D-erythropentofuranosyl)-1,3,5-triazin-2(1H)-one; or 5-aza-2′-deoxy cytidine; and is structurally represented by formula (I).

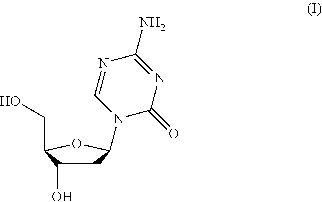

Decitabine, a pyrimidine nucleoside analog of cytidine, is used for treating patients with myelodysplastic syndromes (MDS) including previously treated and untreated, de novo and secondary MDS of all French-American-British subtypes (refractory anemia, refractory anemia with ringed sideroblasts, refractory anemia with excess blasts, refractory anemia with excess blasts in transformation, and chronic myelomonocytic leukemia) and intermediate-1, intermediate-2, and high-risk International Prognostic Scoring System groups.

Decitabine is the active ingredient in the commercially marketed DACOGEN™ product, in the form of a sterile lyophilized powder for injection.

U.S. Pat. No. 3,350,388 discloses a process for the preparation of 2′-deoxy-5-azacytidine, which involves treating a suspension of finely powdered 1-(3,5-di-O-p-toluoyl-2-deoxy-β-D-ribofuranosyl)-4-methylmercapto-2-oxo-1,2-dihydro-1,3,5-triazine in absolute methanol, previously saturated at 0° C. with dry ammonia, and allowing it to stand with occasional stirring in a closed vessel at room temperature for 24 hours. A small amount of the precipitate was removed by filtration and the filtrate was evaporated under reduced pressure. The residue was triturated with absolute ether and then crystallized from anhydrous methanol.

M. W. Winkley et al., in Journal of Organic Chemistry, 35(2), pp. 491-495, 1970 disclose a process for the preparation of 2′-deoxy-5-azacytidine, which involves the condensation of a trimethylsilyl derivative of 5-azacytosine with 2-deoxy-1,3,5-tri-O-acetyl-D-ribofuranose, dissolved in dry ether containing acetyl chloride in acetonitrile, to produce 1-(3,5-di-O-acetyl-2-deoxy-α,β-D-ribofuranosyl)-5-azacytosine, followed by removing the protecting groups with ammonia-saturated ethanol. It also discloses the recrystallization of the β-anomer from a mixture of methanol and 2-propanol to give pure 2′-deoxy-5-azacytidine.

U.S. Pat. No. 3,817,980 discloses a process that involves reacting the bis-silyl compound of 5-azacytosine with 2-deoxy-3,5-di-O-p-toluoyl-ribofuranosyl chloride in the presence of tin tetrachloride. The α/β-anomer mixture of protected decitabine obtained was crystallized from toluene and recrystallized from ethanol. Further, the β-anomer of protected decitabine was obtained by fractional crystallization from ethyl acetate. This β-anomer of protected decitabine was dissolved in absolute methanol saturated with ammonia to form decitabine which was crystallized from ethanol.

Piskala et al., in Journal of Nucleic Acid Research, 1978, 4, 109-113 disclose a process for the preparation of 5-aza-2′-deoxycytidine, which involves the condensation of a compound (A) with silylated 5-azacytosine (B) in acetonitrile at room temperature and in the presence of a molecular sieve to produce a mixture of anomeric di-p-toluate (C) and (D) in high yield, in which the α-anomer strongly predominated. Further, it also discloses a process for the preparation of β-anomer by carrying out the reaction in the presence of a mixture of mercuric oxide and bromide. On methanolysis of protected anomers (C) and (D), the nucleoside 2′-deoxy-5-azacytosine was obtained. This process is depicted in Scheme 1 below.

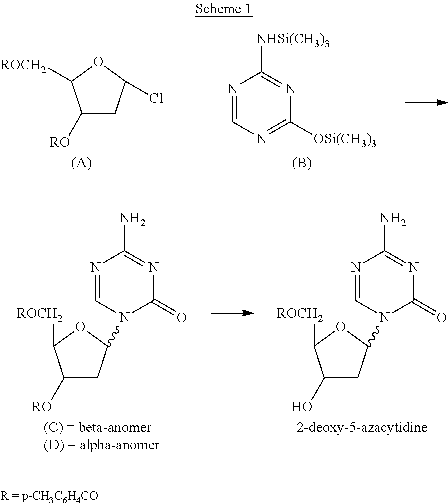

U.S. Pat. No. 4,209,613 discloses a process for preparing a nucleoside in a single step of silylating a nucleoside base, followed by reacting the sugar derivative in the presence of a catalyst.

Jean et al., in Journal of Organic Chemistry, 1986, 51, 3211-3213 disclose a process for the preparation of 5-aza-2′-deoxycytidine, which involves the reaction of methyl 2-deoxy-α,β-D-ribofuranoside with 9-fluorenylmethoxycarbonyl chloride in anhydrous pyridine to give 1-methoxy-3,5-bis(O-Fmoc)-2-deoxyribofuranose. To an ice cold solution of 1-methoxy-3,5-bis(O-Fmoc)-2-deoxyribofuranose, anhydrous HCl gas was passed in dry ether to produce a 1-chloro derivative in situ, which was reacted with the disilylated 5-azacytosine at room temperature in 1,2-dichloroethane, in the presence of catalytic amount of tin chloride. The resulting product mixture contained the two anomers (α and β) in approximately equimolar amounts (α/β=1:0.9). From this mixture, the fully deprotected anomer was obtained by the reaction with triethylamine in dry pyridine followed by crystallization from methanol.

International Application Publication No. WO 2008/101448 A2 (“WO '448”) discloses a process for the preparation of 1-(2-deoxy-alpha-D-erythro-pentofuranosyl)-5-azacytosine (α-anomer of decitabine) by reacting protected 2-deoxy-D-erythro-pentofuranoside (compound A), wherein R¹ represents an alkyl group having 1 to 6 carbon atoms and R² represents an alkanoyl group having 1 to 6 carbon atoms with silylated-5-azacytosine (compound B) wherein R³ represents an alkyl group having 1 to 4 carbon atoms, wherein the substituents R³ can be identical or different, in an inert organic solvent in the presence of a Lewis acid, to form protected 1-(2-deoxy-alpha-D-erythro-pentofuranosyl)-5-azacytosine (compound C) (α-anomer of decitabine) and subsequently removing the alkanoyl protecting groups The process is depicted in Scheme 2 below.

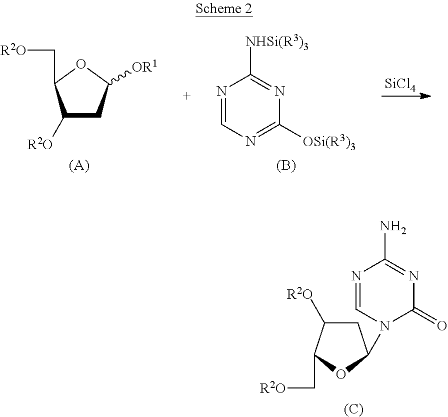

According to the disclosure of WO '448, the prior processes produced only mixtures of α- and β-anomers. Further, the publication discloses that the α-anomer of the protected 1-(2-deoxy-alpha-D-erythro-pentofuranosyl)-5-azacytosine has a lower solubility when compared to the β-anomer, which can be present in small amounts in the crude product and, therefore, it is possible to easily obtain a quite pure product in high yield by crystallization. After removal of the protecting groups by treatment with sodium methoxide in methanol, the α-anomer of decitabine is obtained.

Kun-Tsan et al., in Journal of Pharmaceutical Sciences, 1981, 70(11), 1228-1232, disclose the chemical stability of 5-aza-2′-deoxy cytidine in acidic, neutral and alkaline solution as analyzed by HPLC and possible decomposition products, N-(formylamidino)-N′-β-D-2-deoxyribofuranosylurea and 1-βD-2′-deoxyribofu ranosyl-3-guanyl urea.

Michael et al., in Acta Crystallographica, 1991, C47, 1418-1420 disclose monoclinic crystal coordinates of decitabine monohydrate grown from dimethyl sulphoxide.

International Application Publication No. WO 2004/041195 A1 discloses that decitabine, when exposed to humidity, forms a monohydrate that corresponds to 7% moisture at equilibrium. Decitabine monohydrate is also stable at room temperature and the solid form differential scanning calorimetry of decitabine indicates melting at −201° C., followed by decomposition.

U.S. Patent Application Publication No. 2006/0014949 A1 discloses polymorphic form A, a crystalline anhydrate having an orthorhombic crystal lattice, form B, a crystalline monohydrate, and form C, a crystalline hemihydrate of decitabine. Further, the publication discloses methods for the preparation of form A using solvents such as methanol, acetone, 2-butanol, chloroform, dichloromethane, ethyl ether, hexane, methylsulphide, 2-propanol and 1,1,1-trichloroethane; form B using solvents such as dichloromethane and methanol (1:1), 1,2-dimethoxyethane, 1,1,1,3,3,3-hexafluoro-2-propanol, methanol, methanol and 2,2,2-trifluoroethanol (1:1), 2,2,2-trifluoroethanol, 2,2,2-trifluoroethanol and water (9:1), and water; and form C using 2,2,2-trifluoroethanol and water. Also disclosed is a process for the preparation of amorphous decitabine by crystallization from water.

Despite various process disclosures as discussed above, none of them appears to be particularly suitable for reasons such as not being scalable to industrial quantities, products contaminated with metal impurities, e.g., tin, or other impurities, etc. Hence, there remains a need for simple convenient, industrially amenable, and cost effective processes for the preparation of decitabine.

### SUMMARY

In an aspect, the present application provides processes for the preparation of decitabine, embodiments comprising:

1) silylating 5-azacytosine to give a compound of formula (II),

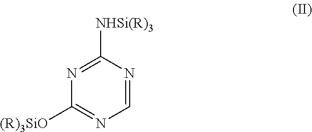

wherein each R independently is an optionally substituted C₁-C₆ alkyl group;

2) coupling the compound of formula (II) with about 0.9 to about 1.2 molar equivalents of a compound of formula (III), per molar equivalent of a compound of formula (II),

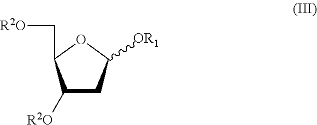

wherein R¹ represents an alkyl group having 1 to 6 carbon atoms and each R² independently is an optionally substituted acyl or aroyl group, in the presence of about 1 to about 1.2 molar equivalents of a non-metallic Lewis acid, per molar equivalent of a compound of formula (II), and an organic solvent, to provide a beta-anomer enriched compound of formula (IV); and

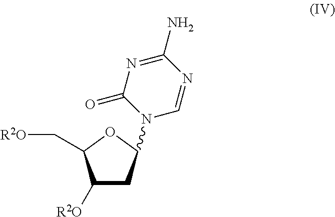

3) deprotecting the compound of formula (IV) to obtain decitabine of formula (I):

In an aspect, the present application provides processes for selectively isolating decitabine, embodiments comprising:

1) providing a solution or suspension of decitabine in an alcohol; and

2) forming a solid, such as by cooling the solution.

In an aspect, the present application provides processes for purifying decitabine, embodiments comprising:

1) providing a solution of decitabine in dimethylsulphoxide; and

2) crystallizing a solid from the solution of 1).

In embodiments, the present application provides processes for preparation of crystalline decitabine having characteristic XRPD peaks located at approximately 7.0, 13.0, 14.3, 18.5, 21.5, and 24.5, ±0.2° 2θ, or in the locations substantially as shown in FIG. 4, comprising:

a) providing a solution of decitabine in dimethylsulphoxide; and

b) crystallizing a solid from the solution of 1) by combining with a methanol and ethyl acetate mixture.

In an aspect, the present application provides substantially pure decitabine.

### BRIEF DESCRIPTION OF THE DRAWINGS

FIG. 1 is an example of an X-ray powder diffraction pattern of crystalline decitabine prepared according to Example 9.

FIG. 2 is an example of a thermogravimetric analysis curve of crystalline decitabine prepared according to Example 9.

FIG. 3 is an example of a differential scanning calorimetry curve of crystalline decitabine prepared according to Example 9.

FIG. 4 is an example of an X-ray powder diffraction pattern of crystalline decitabine prepared according to Example 10.

FIG. 5 is an example of a thermogravimetric analysis curve of crystalline decitabine prepared according to Example 10.

FIG. 6 is an example of an X-ray powder diffraction pattern after the 1^{st} day at room temperature according to Example 11.

FIG. 7 is an example of a thermogravimetric analysis curve after the 1^{st} day at room temperature according to Example 11.

FIG. 8 is an example of an X-ray powder diffraction pattern after the 7^{th} day at room temperature according to Example 11.

FIG. 9 is an example of a thermogravimetric analysis curve after the 7^{th} day at room temperature according to Example 11.

FIG. 10 is an example of an X-ray powder diffraction pattern after the 1^{st} day at 90% relative humidity according to Example 11.

FIG. 11 is an example of a thermogravimetric analysis curve after the 1^{st} day at 90% relative humidity according to Example 11.

FIG. 12 is an example of an X-ray powder diffraction pattern after the 7^{th} day at 90% relative humidity according to Example 11.

FIG. 13 is an example of a thermogravimetric analysis curve after the 7^{th} day at 90% relative humidity according to Example 11.

### DETAILED DESCRIPTION

Unless otherwise specified, the term “decitabine” refers to the beta-anomer of 4-amino-1-(2-deoxy-D-erythropentofuranosyl)-1,3,5-triazin-2(1H)-one.

The term “beta-anomer enriched” refers to a compound containing more than about 50% of the beta-anomer, while the other anomer being less than about 50%.

In an aspect, the present application provides processes for the preparation of decitabine, embodiments comprising:

1) silylating 5-azacytosine to give a compound of formula (II),

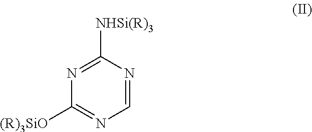

wherein each R independently is an optionally substituted C₁-C₆ alkyl group;

2) coupling the compound of formula (II) with about 0.9 to about 1.2 molar equivalents of a compound of formula (III), per molar equivalent of a compound of formula (II),

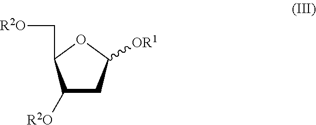

wherein R¹ represents an alkyl group having 1 to 6 carbon atoms and each R² independently is an optionally substituted acyl or aroyl group, in the presence of about 1 to about 1.2 molar equivalents of a non-metallic Lewis acid, per molar equivalent of a compound of formula (II), and an organic solvent, to provide a beta-anomer enriched compound of formula (IV); and

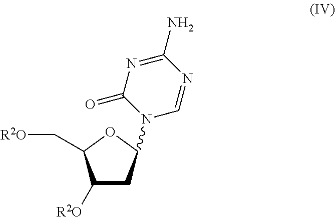

3) deprotecting the compound of formula (IV) to obtain decitabine.

These steps for the process are separately described below.

Step 1).

The compound of formula (II) may be obtained by reacting 5-azacytosine with a silylating agent, optionally in the presence of a catalyst.

Silylating agents that may be used in the processes of the present application include, but are not limited to, hexamethyldisilazane (HMDS), trimethylsilyl chloride (TMSCl), t-butyldimethylsilyl chloride, and mixtures thereof.

Catalysts that may be used include, but are not limited to, ammonium sulphate and ammonium phosphate.

The amounts of silylating agent that may be used range from about 1 to 2.5 molar equivalents, per molar equivalent of 5-azacytosine. In a particular embodiment, about 2 molar equivalents, per molar equivalent of 5-azacytosine is used.

The process is carried out using an organic solvent. The organic solvents that may be used in the silylation reaction include, but are not limited to, nitrile solvents such as acetonitrile, propionitrile, and the like.

The silylation reaction may be carried out at temperatures up to the boiling point of the solvent, when an organic solvent is used. In one of the particular embodiments, the silylation reaction may be carried out using HMDS and ammonium sulphate, in the presence of acetonitrile, at temperatures about 75-85° C.

The compound of formula (II) may be used in situ for coupling with the compound of formula (III). Optionally, the compound of formula (II) may be isolated before coupling with the compound of formula (III), using techniques known in the art.

Step 2).

Step 2) involves coupling the compound of formula (II) with about 0.9 molar equivalent to about 1.2 mole equivalents of a compound of formula (III), per molar equivalent of a compound of formula (II), wherein R¹ represents an alkyl group having 1 to 6 carbon atoms and each R² independently is an optionally substituted acyl or aroyl group.

In a specific embodiment, R¹ is methyl and each R² is acetyl. The compound of formula (III) may be obtained by methods known in the art.

In an embodiment, about 1 molar equivalent, or about 1.2 molar equivalent, of a compound of formula (III), per molar equivalent of 5-azacytosine, is used.

Non-metallic Lewis acids that may be used for coupling include, but are not limited to, trifluoromethanesulfonic (“triflic”) acid, trimethylsilyl triflate (TMS triflate), triisopropylsilyl triflate, and the like.

The amounts of the non-metallic Lewis acid that may be used range from about 1 to about 1.2 molar equivalents, per molar equivalent of 5-azacytosine used in step 1). In embodiments, about 1 mole, or about 1.05 mole, of non-metallic Lewis acid, per molar equivalent of 5-azacytosine, is used.

Organic solvents that may be used in the coupling step include, but are not limited to: halogenated hydrocarbons such as dichloromethane, 1,2-dichloroethane, chloroform, and carbon tetrachloride; esters such as ethyl acetate, n-propyl acetate, n-butyl acetate, and t-butyl acetate; nitriles such as acetonitrile and propionitrile; and toluene.

In a particular embodiment, the process involves the condensation of methyl-2-deoxy-3,5-di-O-acetyl-D-erythro-ribofuranoside with silylated 5-azacytosine, in the presence of TMS triflate and dichloromethane, to provide a beta-anomer enriched 1-(2-deoxy-3,5-di-O-acetyl-D-erythro-ribofuranosyl)-5-azacytosine.

The coupling reaction may be carried out at temperatures ranging from about 0° C. to about 45° C., or at about 25° C. to about 30° C.

After completion of the reaction, the reaction mixture may optionally be cooled to temperatures in the range of 10-15° C. and the reaction may optionally be quenched by the addition of a base, such as sodium carbonate, sodium bicarbonate, potassium carbonate, and mixtures thereof, either as a solid or as a solution in water.

In embodiments, the base is used as a solution in water. The amount of base that may be used depends upon the desired pH range to which the reaction mass is to be adjusted. In embodiments, the pH of the reaction mass is adjusted to be in the range of about 6-8. The concentration of base in an aqueous solution may be in the range of about 2% to about 10%. However, the desired concentration may vary depending upon the process requirements.

In particular embodiments, about 3% aqueous sodium carbonate is used for quenching the reaction.

Optionally, the reaction may be quenched with a basic ion-exchange water insoluble resin.

After quenching the reaction, the layers are separated, and the aqueous layer may be optionally extracted with an organic solvent, for example, dichloromethane, and the organic layer obtained may be distilled under vacuum to isolate the product.

Optionally, the product obtained may be purified by crystallization from organic solvents including, but not limited to, alcohols such as methanol, ethanol, isopropanol, and the like.

The inventors of the present application have found that the compound of formula (IV);

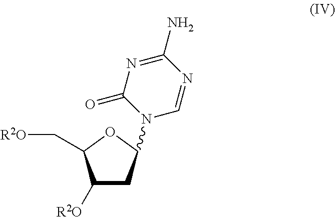

prepared by the conditions such as the molar ratios of compounds (II) and (III) and the temperature range as described above results in a beta-anomer enriched product.

The term “beta-anomer enriched” refers to a compound having at least about 50% of the beta-anomer, with the other anomer being less than about 50%.

Step 3).

Step 3) involves a removal of the acyl or aroyl protecting groups of the compound of formula (IV) obtained in step 2), by reacting with a base in the presence of an organic solvent, to obtain decitabine

Suitable bases that may be used for the removal of the acyl or aroyl protecting groups include, but are not limited to: alkali metal hydroxides such as sodium hydroxide, potassium hydroxide; metal alkoxides such as sodium methoxide and sodium ethoxide; ammonia; organic bases such as diethylamine, butylamine, and propylamine; and the like.

Solvents that may be used include, but are not limited to, C₁-C₄ alcohols such as methanol, ethanol, isopropanol, and mixtures thereof.

In a particular embodiment, methanolic ammonia is utilized for the removal of acyl or aroyl protecting groups.

In a further embodiment, about 2 to 40 equivalents of methanolic ammonia, per molar equivalent of compound of formula (IV), may be used for removal of acyl or aroyl protecting groups.

The temperatures at which the reaction may be carried out range from about 5° C. to about 45° C. In embodiments, the reaction may be carried out at temperatures about 20° C. to about 35° C.

After completion of the reaction, decitabine may be isolated by optionally cooling the reaction mass to lower temperatures, such as about 0-15° C., or about 0-5° C., maintaining for sufficient period of time and filtering the formed solid. The solid thus obtained may be optionally dried at a desired temperature.

The inventors of the present application have found that prior processes for the preparation of decitabine, involving the removal of the acyl or aroyl protecting groups of a compound of formula (IV), resulted in decitabine having unacceptable amounts, such as more than about 0.5% by weight, of mono acyl or aroyl protected decitabine.

The inventors of the present application have further found that the use of higher quantities of base results in the reduction of the undesired impurities like mono acyl- or aroyl-protected decitabine

In embodiments, the present application provides processes for preparing decitabine substantially free of mono acyl- or aroyl-protected decitabine of the formula (V),

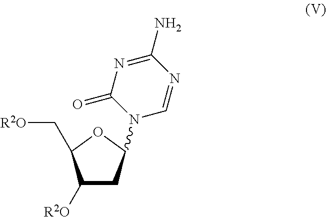

wherein each R² independently is hydrogen, or an acyl or an aroyl group, with the proviso that both are not hydrogen, acyl, or aroyl, embodiments comprising deprotecting the compound of formula (IV) in the presence of excess ammonia.

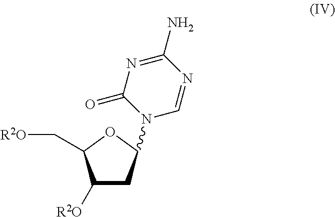

In embodiments, excess ammonia includes about 2 to 40 equivalents of ammonia, or 25-40 equivalents of ammonia, per molar equivalent of a compound of formula (IV).

In a further embodiment, the present application provides processes for selectively isolating decitabine, embodiments comprising:

1) providing a solution or suspension of decitabine in an alcohol;

2) forming a solid, such as by cooling the solution.

Step 1) involves providing a solution or suspension of decitabine in an alcohol. The solution or suspension of decitabine in step 1) may be provided from a reaction by which the compound is synthesized, or by combining decitabine with an alcohol.

The decitabine used in step 1) may be a mixture of α- and β-anomers, wherein the α- to β-anomer ratios may range from about 1:1 to a β-anomer enriched product.

Alcohols used for preparing a solution or suspension of decitabine include, but are not limited to, methanol, ethanol, and isopropanol.

Optionally, the solution or suspension of decitabine may be provided in the presence of a base.

Suitable bases that can be used include, but are not limited to: alkali metal alkoxides such as sodium methoxide and sodium ethoxide; ammonia; organic bases such as diethylamine, butylamine, and propylamine; and the like. In a particular embodiment, 10% methanolic ammonia is used.

The temperatures at which the solution or suspension is provided may range from about 0° C. to about 50° C., or about 25° C. to about 35° C.

The solution or suspension may be stirred for about 5 minutes to 6 hours, or longer, depending upon the reaction batch size and other process conditions.

Step 2) involves forming a solid from the solution.

The solution or suspension obtained in step 1) may optionally be cooled to a desired temperature, which facilitates selectively obtaining decitabine.

In embodiments, the solution or suspension obtained in step 1) may be cooled to temperatures in the range of about 0° C. to about 25° C., or about 10° C. to about 15° C., or about 0° C. to about 5° C. Further, the temperatures to which the solution or suspension is cooled may also depend on the solvent used in step 1).

Optionally, the solution or suspension may be stirred for about 5 minutes to 3 hours, or longer, depending upon the desired extent of selective isolation of decitabine.

The solid product may be isolated by methods known in the art. For example, the product may be isolated by distilling solvent from the mass, or by filtration. The product thus obtained may optionally be further purified using methods known in the art or by a process as disclosed in the present application.

The processes of the present application provides decitabine having purity of at least about 90%, or at least about 95%, or at least about 99%, by weight, as determined using high performance liquid chromatography (HPLC).

An embodiment of the preparation of decitabine according to the above description is schematically summarized as Scheme 3.

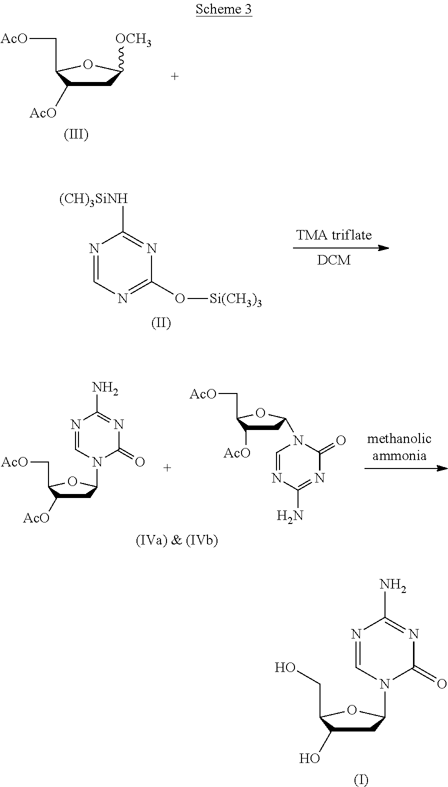

In an embodiment, the present application provides processes for the purification of decitabine, embodiments comprising:

1) providing a solution of decitabine in dimethylsulphoxide; and

2) crystallizing a solid from the solution.

Step 1).

The solution of decitabine may be provided by the dissolution of decitabine in dimethylsulphoxide, or may be obtained form a process by which the compound is synthesized.

Any polymorphic form of decitabine, such as any crystalline or amorphous form of decitabine obtained by any method, is acceptable for providing the solution.

The decitabine used may be a mixture of α- and β-anomers, wherein the α- to β-anomer ratios may range from about 1:1 to a β-anomer enriched compound by weight.

The concentration of decitabine in the solution is not critical as long as sufficient solvent is employed to ensure total dissolution. The amount of solvent employed is typically kept to a minimum to avoid excessive product losses during crystallization and isolation. The quantities of solvents used for providing solutions of decitabine may range from about 5 times to about 10 times, or more, the weight of decitabine.

The solutions may be prepared at temperatures ranging from about 0° C. to about 35° C., or the boiling point of the solvent chosen. Depending on the solvent and the quantity used, solute may dissolve at 25-35° C., or the solution may need to be heated to higher, including reflux, temperatures.

The solution may optionally be filtered by passing through paper, glass, fibre, or other membrane material, or a bed of clarifying agent such as Hyflow (flux-calcined diatomaceous earth). Depending upon the equipment used and the concentration and temperature of the solution, the filtration apparatus may need to be heated to avoid premature crystallization.

Optionally, the solution of decitabine in the dimethylsulphoxide may be filtered more than once to reduce the ash content in the final product.

In a particular embodiment, a solution of decitabine in dimethylsulphoxide may be filtered three times through a Hyflow bed, resulting in reduction of the ash content from about 0.1% to about 0.04%, by weight.

Step 2).

Crystallizing a solid from the solution of step 1) may be performed by methods such as cooling, partial removal of the solvent from the mixture, seeding, combining an anti-solvent with the solution, or any combinations thereof.

In embodiments, the crystallization may be carried out by combining the solution of step 1) with a suitable anti-solvent. Adding the solution of step 1) to an anti-solvent, or adding an anti-solvent to the solution of step 1), to initiate crystallization process are both within the scope of the present application.

In embodiments, an alcohol, an ester, or any mixtures thereof, is used as an anti-solvent.

The esters used include, but are not limited to, ethyl acetate, n-propyl acetate, n-butyl acetate, t-butyl acetate, and the like.

The alcohols used include, but are not limited to, C₁-C₄ alcohols such as methanol, ethanol, isopropanol, n-propanol, and the like.

In particular embodiments, a mixture of an ester and alcohol is used as an anti-solvent.

The decitabine solution can be combined with the desired anti-solvent at temperatures ranging from about 0° C. to 40° C., or higher, or from about 20-30° C. The combination may be carried out for a sufficient period of time, which may range from about 5 minutes to about 3 hours, or longer, to effect the desired extent of crystallization.

The quantities of anti-solvent used for crystallization of decitabine can range from about 5 to 70 times, or about 20 to 55 times, the weight of decitabine used.

The temperatures at which a solid precipitates depend on the solvents and their quantities used. Optionally, a suspension obtained may be cooled to the desired temperature and may then be stirred for about 30 minutes to 5 hours, or longer, depending upon the desired extent of precipitation.

The obtained precipitate may be isolated using conventional techniques. One skilled in the art will appreciate that there are many ways to separate a solid from a slurry, for example it may be separated by using any techniques such as filtration by gravity or by suction, centrifugation, decantation, and the like. After separation, the solid may optionally be washed with a suitable solvent.

The isolated solid may optionally be further dried. Drying may be suitably carried out in a tray dryer, vacuum oven, air oven, fluidized bed dryer, spin flash dryer, flash dryer, and the like. The drying may be carried out at temperatures about 35° C. to about 100° C., or about 50° C. to about 70° C., optionally under reduced pressure. The drying may be carried out for any time periods to obtain a desired purity, such as from about 1 to about 25 hours, or longer, or for a time period about 5 to 8 hours.

Decitabine obtained by the processes of the present application may be characterized by any one or more of its X-ray powder diffraction (“XRPD”) pattern, differential scanning calorimetry (DSC) curve, and thermogravimetric analysis (TGA) curve. XRPD data reported herein were generated with a Bruker Axe D8 Advance Powder X-ray Diffractometer, using copper Ka radiation.

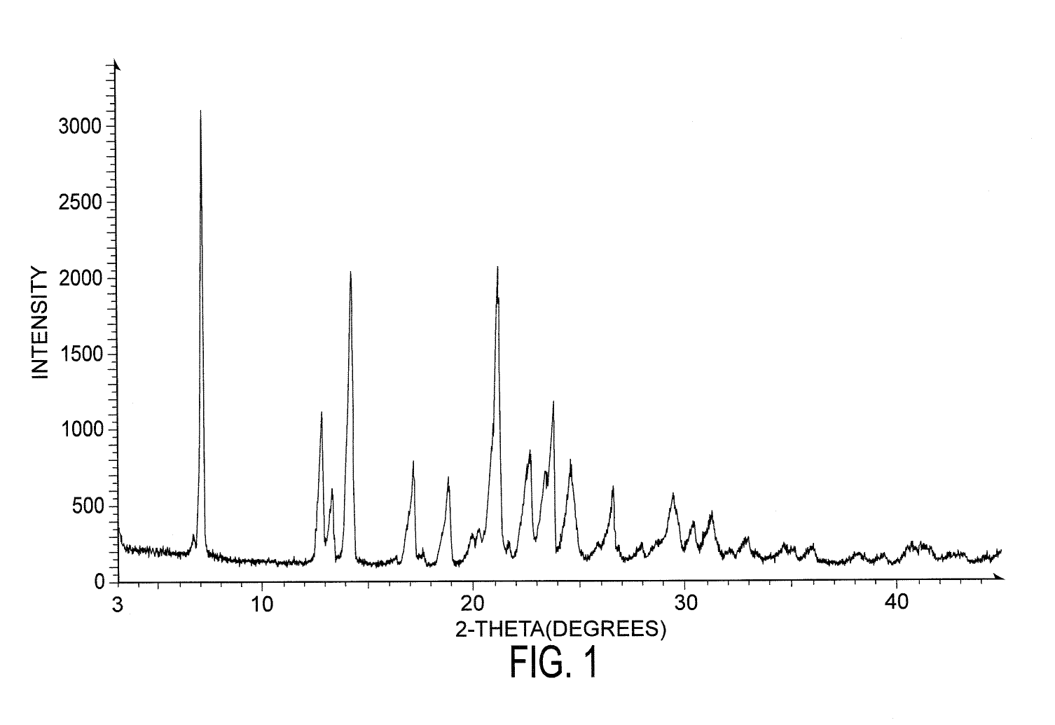

Decitabine obtained by an embodiment of the present application may be characterized by an XRPD diffraction pattern having characteristic peaks located substantially in accordance with the pattern of FIG. 1.

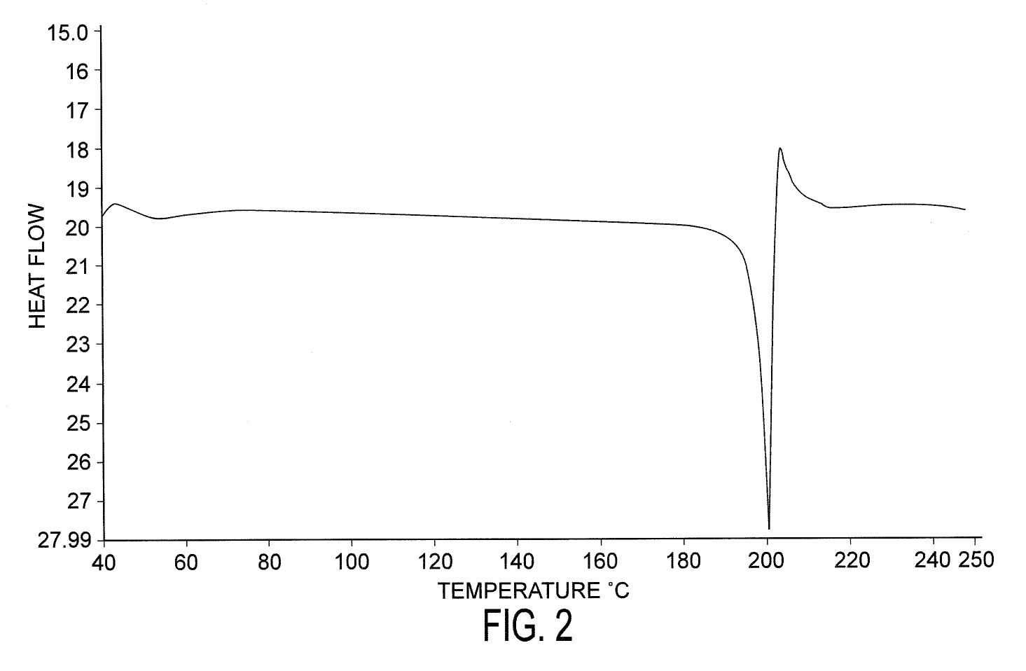

Decitabine obtained by an embodiment of the present application may be characterized by its differential scanning calorimetry curve, having a peak at about 200° C., or by its DSC curve substantially as shown in FIG. 2. In an analysis, the decitabine has been observed to decompose using a 10° C./minute heating rate and a starting temperature of 40° C. in a DSC Q1000 model instrument. Thus, the DSC event resulted from decomposition of decitabine.

Thermogravimetric analyses reported herein for decitabine were carried out using a SHIMADZU, DTG-60, Thermogravimetric Analyzer with a ramp of 10° C./minute up to 250° C.

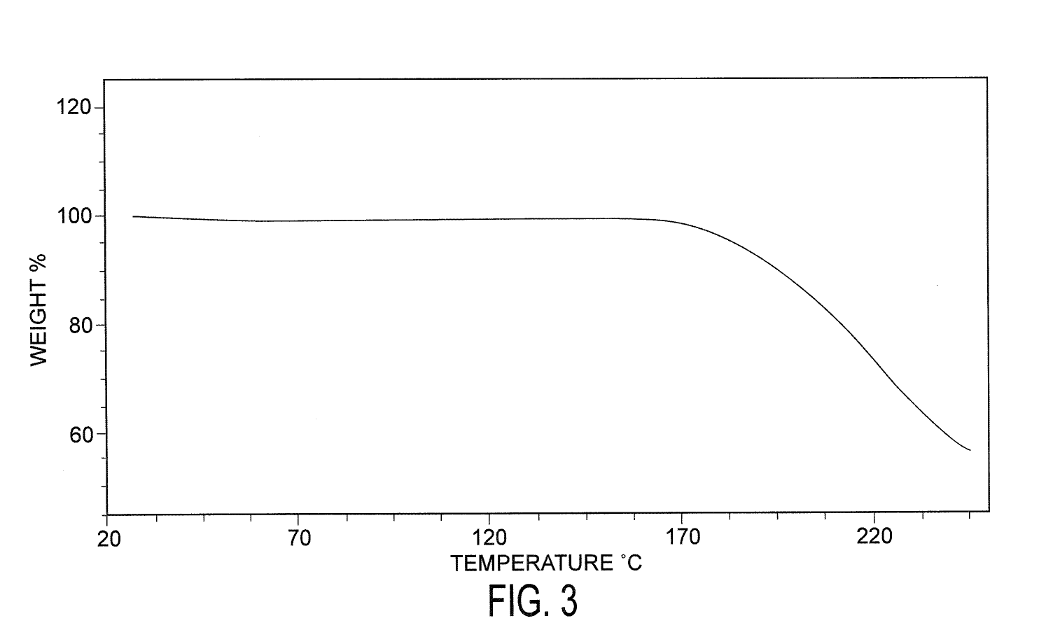

Decitabine obtained by an embodiment of the present application may be characterized by thermogravimetric analysis weight loss of less than about 1%, and/or by a TGA curve substantially as shown in FIG. 3.

The polymorphic form of decitabine obtained by the processes of the present application, when exposed to the atmosphere, absorbs water and is transformed to a monohydrate form, with an X-ray diffraction pattern substantially as shown in FIG. 1.

In a specific aspect, the present application provides processes for the preparation of decitabine, comprising:

1) silylating 5-azacytosine in acetonitrile to give a compound of formula (II),

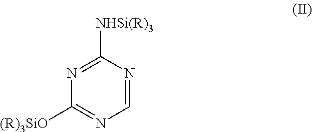

wherein each R independently is an optionally substituted C₁-C₆ alkyl group;

2) coupling the compound of formula (II) with about 0.9 to about 1.2 molar equivalents of a compound of formula (III), per molar equivalent of a compound of formula (II),

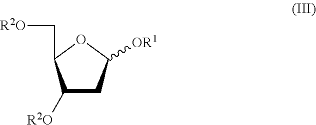

wherein R¹ represents an alkyl group having 1 to 6 carbon atoms and each R² independently is an optionally substituted acyl or aroyl group, in the presence of about 1 to about 1.2 molar equivalents of trimethylsilyl triflate, per molar equivalent of a compound of formula (II), and dichloromethane, to provide a beta-anomer enriched compound of formula (IV);

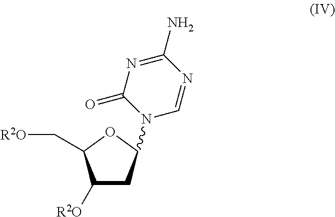

3) deprotecting the compound of formula (IV) by reacting with methanolic ammonia, to obtain decitabine;

4) providing a solution of decitabine obtained from step 3) in dimethylsulphoxide; and

5) crystallizing a solid from the solution of 1) by combining with a mixture of methanol and ethylacetate mixture.

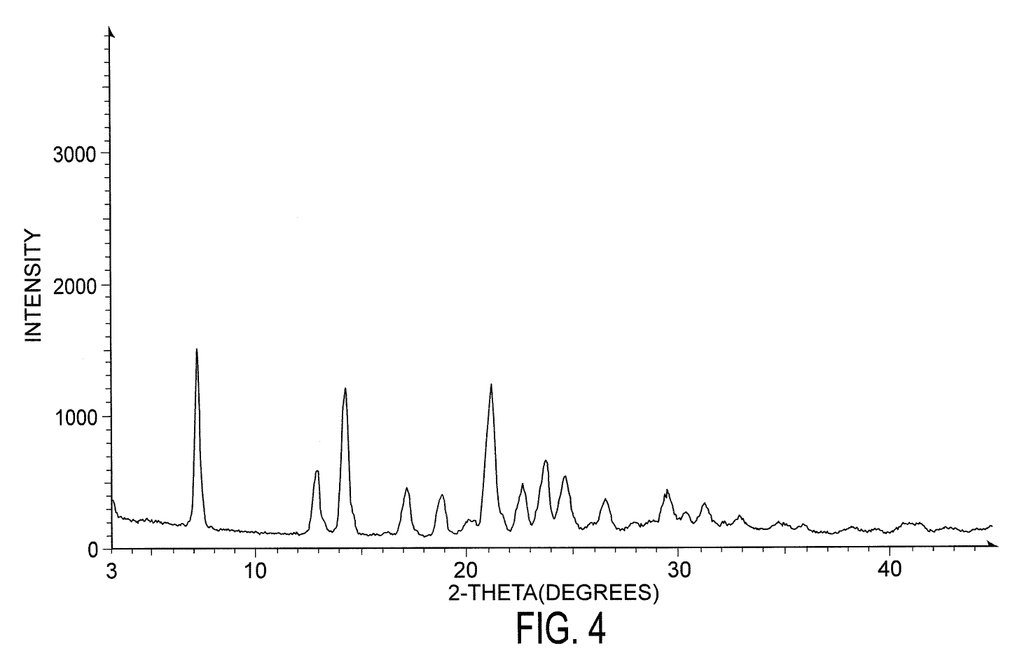

In embodiments, the present application provides processes for preparing crystalline decitabine having characteristic XRPD peaks located at approximately 7.0, 13.0, 14.3, 18.5, 21.5 and 24.5±0.2° 2θ, or having peaks located substantially as shown in FIG. 4, comprising:
a) providing a solution of decitabine in dimethylsulphoxide; and
b) crystallizing a solid from the solution by combining with a mixture of methanol and ethyl acetate.

Decitabine obtained by processes of the present application from dimethylsulphoxide solutions, using a methanol-ethyl acetate anti-solvent, when carried out in closed conditions or when carried out in an isolator, is an anhydrous crystalline form of decitabine with a PXRD pattern substantially in accordance with FIG. 4.

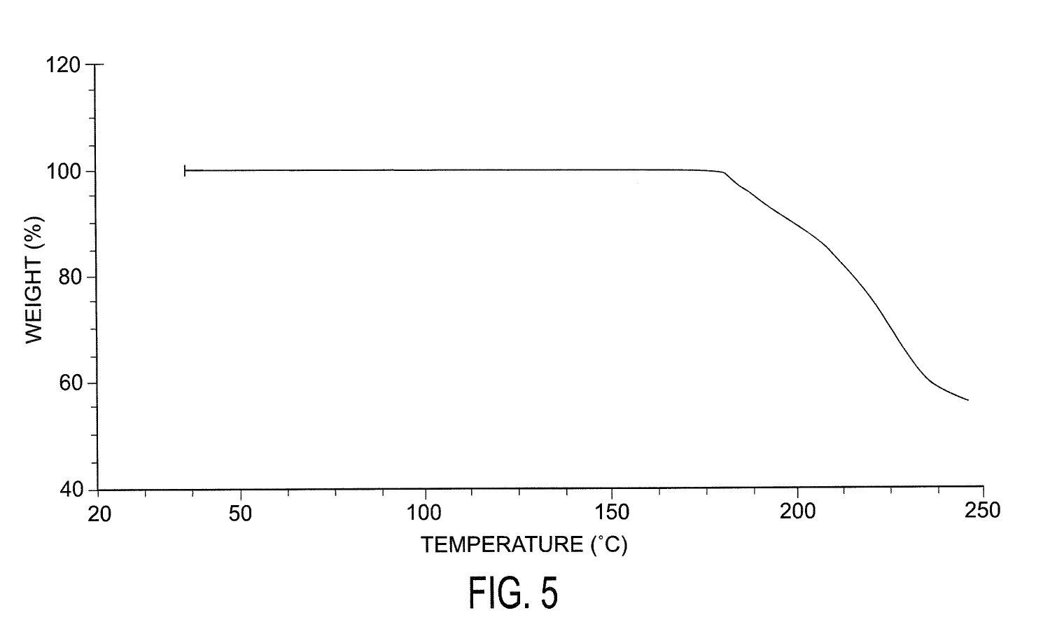

Decitabine obtained may be further characterized by thermogravimetric analysis weight loss of less than about 1%, or less than about 0.25%, or less than about 0.1%, and/or by a TGA curve substantially as shown in FIG. 5.

It is observed that, when crystallized decitabine is exposed to atmospheric humidity, the anhydrous form may be converted to a monohydrate form of decitabine. An illustrative example of the result of such conversion can be shown by a PXRD pattern as given in FIG. 1.

In embodiments, the present application provides a storage system for decitabine. A storage system of the present application comprises at least one sealed polymeric bag, (e.g., a transparent or opaque polyethylene bag having a thickness of about 0.1 mm to about 0.5 mm) or a combination of such bags, which, if desired, may be sealed inside a laminated aluminum bag. An oxygen absorbent and a moisture absorbent (or desiccant), may be included between one or more of such bags. Finally, the packaged material is stored in sealed HDPE containers.

In a specific embodiment, the present application provides a storage system for decitabine, comprising:

a) one or more sealed polymeric bags containing decitabine;

b) an external sealed polymeric bag containing the bag or bags of a), optionally sealed inside a laminated aluminum bag; and

c) an oxygen absorbent and optionally a moisture absorbent disposed between the bag or bags of a) and the bag of b).

Suitable oxygen absorbents include but are not limited to organic types, based on ascorbic acid, and inorganic types, based on iron powder-containing materials. Commercial products Ageless Z200 and Ageless Z100, and the like, may be utilized as oxygen absorbents for maintaining the stability of decitabine.

Suitable desiccants include, but are not limited to, aluminum oxide, calcium chloride, CaSO₄ (Drierite™), molecular sieves (e.g., activated molecular sieves), silica gel, and the like, and any combinations thereof.

Anhydrous crystalline decitabine with an initial XRD pattern substantially in accordance with FIG. 4 remains stable for a period at least about 4 months, or about 6 months, when stored at 40±2° C. and a relative humidity of 75±5% under the packaging conditions described above. The TGA weight losses of representative samples stored under these conditions are tabulated below.

| Storage Time | Weight Loss (%) |
|---|---|
| 1 month | 0.07 |
| 2 months | 0.15 |
| 3 months | 0.26 |
| 4 months | 0.16 |

The processes of the present application for the selective isolation or purification of decitabine may be optionally be repeated to get substantially pure decitabine having a purity greater than or equal to about 99.5%, or greater than or equal to about 99.8%, by weight as determined using high performance liquid chromatography (HPLC).

The present application includes substantially pure decitabine, wherein the amount of any individual process-related impurity listed in Table 1 is less than about 0.15%, or less than about 0.1%, by weight, and/or the sum of all of these impurities present is less than about 0.2%, by weight.

TABLE 1

| Impurity | Formula |
|---|---|
| A | 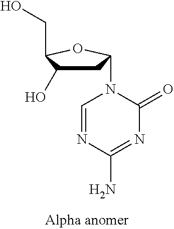 |
| B | 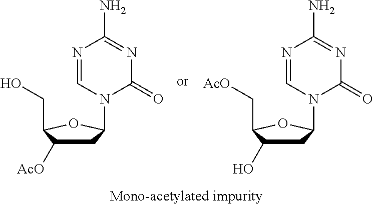 |
| C | 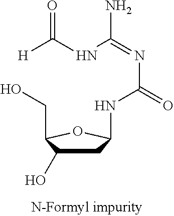 |
| D | 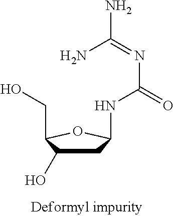 |
| E | 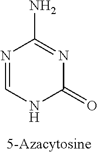 |
| F | 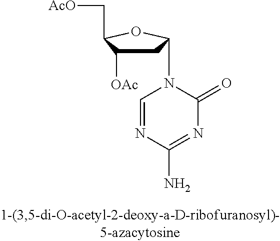 |
| G | 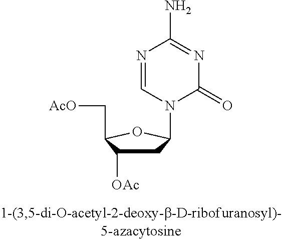 |

The term “substantially free” with respect to drug-related impurities, as used herein shall be understood to mean decitabine with little or no content of the impurities. Hence, a substantially pure decitabine has relatively minor amounts of any impurity of decitabine resulting from the process of the preparation, e.g., less than about 0.15 weight percent, or less than about 0.1 weight percent, or less than about 0.05 weight percent.

In particular embodiments, substantially pure decitabine is decitabine containing less than about 0.2% by weight of total combinations of the impurities of Table 1, and it may be produced by processes of the present application.

The impurities may be analyzed using various methods. Representative useful HPLC methods are described below.

Method I.

Column: Inertsil ODS-3V, 250×4.6, 5 μm or equivalent.

Buffer: 2.72 g of potassium dihydrogen phosphate dissolved in 1000 mL of water, pH adjusted to 6.8 with triethylamine.

Mobile Phase A: Mixture of buffer and methanol in the ratio 95:5 by volume.

Mobile Phase B: Mixture of buffer and acetonitrile in the ratio 50:50 by volume.

Flow rate: 1.0 mL/minute.

Detector wavelength: 210 nm.

Temperature: Ambient.

Sample size: 10

Diluent: Mobile phase A.

Mobile phase gradient program:

| Minutes | % A | % B |
|---|---|---|
| 0.01 | 100 | 0 |
| 5.0 | 100 | 0 |
| 40.0 | 40 | 60 |
| 55.0 | 40 | 60 |
| 57.0 | 100 | 0 |
| 65.0 | 100 | 0 |

Method II.

Column: Inertsil ODS, 250×4.6, 5 μm or equivalent.

Buffer: 2.72 g of potassium dihydrogen phosphate dissolved in 1000 mL water, pH adjusted to 6.8 with triethylamine.

Mobile Phase A: Mixture of buffer and methanol in the ratio 95:5 by volume.

Mobile Phase B: Mixture of buffer and acetonitrile in the ratio 50:50 by volume.

Flow rate: 1.0 mL/minute.

Detector wavelength: 242 nm.

Temperature: Ambient.

Sample size: 20 μL.

Diluent: Dimethylsulphoxide.

Mobile phase gradient program:

| Minutes | % A | % B |
|---|---|---|
| 0.01 | 100 | 0 |
| 5 | 100 | 0 |
| 40 | 40 | 60 |
| 55 | 40 | 60 |
| 57 | 100 | 0 |
| 65 | 100 | 0 |

Using these methods, relative retention times (decitabine=1) listed in Table 2 are obtained for certain process-related impurities and degradation products.

TABLE 2

| Impurity | RRT |
|---|---|
| Impurity A | ~0.81 (Method I) |
| Impurity B | ~3.19 (Method I) |
| Impurity C | ~2.27 (Method II) |
| Impurity D | ~0.52 (Method I) |
| Impurity E | ~0.42(Method I) |
| Impurity F | ~3.78 (Method I) |
| Impurity G | ~4.27 (Method I) |

The compendial upper limit for inorganic impurities, called “residue on ignition” (ROI), in an active pharmaceutical ingredient is a small amount. Since silicon is a metalloid, which will be reflected in a residue on ignition test, silicon-containing groups have to be removed to meet the specification. A procedure for performing this analysis is provided as Test 281 “Residue on Ignition,” in United States Pharmacopeia 29, United States Pharmacopeial Convention, Inc., Rockville, Md., 2005.

The inventors of the present application have found that processes for the purification of decitabine as descried in the present application reduce the ROI content to less than about 0.05% w/w, or to less than about 0.03% w/w.

The present application also relates to methods of preparing decitabine to reduce the content of residual solvents. A method for reducing the non-volatile solvent content comprises providing a suspension of decitabine in a solvent comprising esters, alcohols, ethers, or mixtures thereof, maintaining for a sufficient period of time to reduce the amount of organic volatile impurities, and recovering decitabine containing less than or about 3000 ppm of non-volatile solvents, using solvents such as esters, alcohols, ethers and the like. In embodiments, the obtained decitabine is in pure crystalline form.

As used herein, the term “non-volatile” refers to organic solvents having a boiling point at least about 140° C. Examples of such solvents include, but are not limited to, dimethylsulphoxide and N,N-dimethylformamide.

Certain specific aspects and embodiments of the present application will be explained in more detail with reference to the following examples, which are provided only for purposes of illustration and should not be construed as limiting the scope of the application in any manner.

### EXAMPLES

### Example 1

### 1-Methoxy-2-deoxyribose (II)

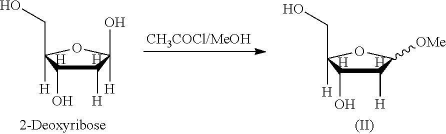

Methanol (8 L) is charged into a reactor, stirred for 10-15 minutes, and cooled to 0-5° C., then acetyl chloride (0.16 Kg) is added and the mixture is stirred for 10-15 minutes at 0-5° C. The obtained methanolic hydrogen chloride is transferred into another container.

Methanol (40 L) is charged into a clean reactor and stirred for 10-15 minutes at 25-35° C. 2-deoxy-D-ribose (4.0 Kg) is charged into the reactor and the mixture is stirred at 25-35° C. for 10-15 minutes. The mass is cooled to 0-5° C. and the methanolic hydrogen chloride solution prepared above is charged into the reactor at same temperature. The obtained mass is maintained at 0-5° C. for 2-3 hours. Sodium bicarbonate (1.6 Kg) is charged into the mass at 0-5° C. and the mass is filtered through a Hyflow bed. The filtrate is collected in another container and the filter bed is washed with methanol (8 L). The combined filtrate is distilled completely under vacuum below 50° C. to afford 1-methoxy-2-deoxyribose. Yield: 4.4 Kg (99.55%).

### Example 2

### 1-Methoxy 3,5-di-O-acetyl-D-ribofuranose (III)

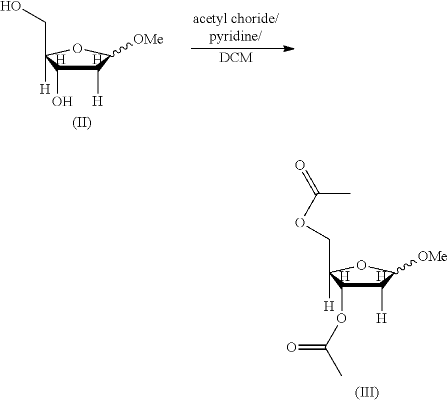

Dichloromethane (60 L) is charged into a reactor and 1-methoxy-2-deoxyribose (4.0 Kg), obtained from Example 1, is added. The mixture is stirred for 10-15 minutes at 25-35° C. and cooled to 0-5° C. Pyridine (10.68 Kg) is added at 0-5° C. and stirred for 10-15 minutes. Acetyl chloride (1.06 Kg) is added to the mass slowly over 15-30 minutes at 0-5° C. A second quantity of acetyl chloride (1.06 Kg) is added to the mass slowly over 15-30 minutes at 0-5° C. A third quantity of acetyl chloride (8.48 Kg) is added to the mass slowly over 15-30 minutes at 0-5° C.

The mass is stirred for 0-5° C. for about 3 hours then is filtered and the filtrate is collected into another container. The reactor is washed with dichloromethane (20 L) and the filter bed is washed with this dichloromethane. Hydrochloric acid (36%, 2.01 L) is charged into a container and water (38 L) is added, then the mixture is stirred for 10-15 minutes to obtain hydrochloric acid solution. The total filtrate is charged into a reactor and cooled to 15-20° C. Half of the hydrochloric acid solution prepared above is added slowly at 15-20° C. and the mixture is stirred at 15-20° C. for 10-15 minutes. The mass is allowed to settle at 15-20° C. for 10-15 minutes and the layers are separated. The organic layer is charged into a reactor and cooled to 15-20° C. The remaining quantity of hydrochloric acid solution prepared above is added slowly to the organic layer in the reactor at 15-20° C. and the mixture is stirred at 15-20° C. for 20-30 minutes. The mass is allowed to settle at 15-20° C. for 10-15 minutes, and then the layers are separated.

Sodium carbonate (2.01 Kg) and water (40 L) are combined and stirred for 20-30 minutes. Half of the sodium carbonate solution is added to the organic layer and the mixture is stirred at 25-35° C. for 20-30 minutes. The mass is allowed to settle at 25-35° C. for 10-15 minutes. The layers are separated, remaining sodium carbonate solution is added to the organic layer, and the mixture is stirred at 25-35° C. for 20-30 minutes. The mass is allowed to settle at 25-35° C. for 10-15 minutes and the layers are separated. The organic layer is charged into a reactor and water (20 L) is added. The mixture is stirred at 25-35° C. for 20-30 minutes. The mass is allowed to settle at 25-35° C. for 10-15 minutes. The layers are separated. The organic layer is charged into a clean reactor, water (20 L) is added, and the mixture is stirred at 25-35° C. for 15-20 minutes. After the mass settles at 25-35° C. for 10-15 minutes, the layers are separated. The organic layer is charged into a reactor, water (20 L) is added, and the mixture is stirred at 25-35° C. for 10-15 minutes. The layers are separated and the organic layer is distilled completely under vacuum below 50° C. to obtain 1-methoxy-3,5-di-O-acetyl-D-ribofuranose. Yield: 4.9 Kg (78.1%).

### Example 3

### 1-(3,5-di-O-acetyl-2-deoxy-D-ribofuranosyl)-5-azacytosine

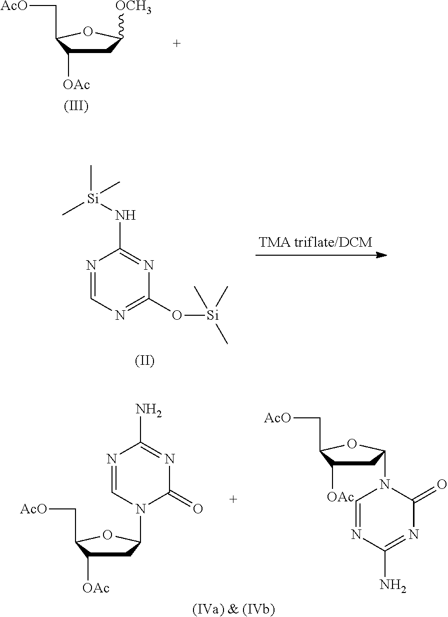

5-Azacytosine (10 g) and acetonitrile (50 mL) are charged into a round bottom flask at 25-35° C. and stirred for 5-10 minutes. Ammonium sulphate (0.49 g) and HMDS (38 mL) are added and the mixture is heated to 60-80° C., maintained for 10-15 minutes, and stirred at 75-80° C. for 2 hours. The solvent is distilled completely under vacuum at 75-80° C. for 20-30 minutes, and dichloromethane (230 mL) and 1-methoxy 3,5-di-O-acetyl-D-ribofuranose (20.71 g) are added to the residue and stirred for 5-10 minutes at 25-30° C. The mass is cooled to 0-5° C. and TMS triflate (16.59 mL) is added and stirred for 10-15 minutes at 0-10° C. The mass is gradually heated to 25-30° C. and stirred for 1-1.5 hours. The mass is cooled to 10-20° C. and the reaction is quenched by adding 3% sodium carbonate solution (230 mL), then the mixture is stirred for 5-10 minutes and layers are separated at 18-25° C. The aqueous layer is extracted with dichloromethane (2×230 mL). The organic layers are combined and distilled at 35-45° C. for 35-40 minutes. Yield: 20.0 g (71.94%); Purity: 52.87% (β-anomer).

### Example 4

### 1-(3,5-di-O-acetyl-2-deoxy-D-ribofuranosyl)-5-azacytosine

5-Azacytosine (20 g) and acetonitrile (100 mL) are charged into a round bottom flask at 25-35° C. and stirred for 5-10 minutes. Ammonium sulphate (0.94 g) is added and stirred for 5 to 10 minutes. HMDS (74.8 mL) is added and the mass is heated to 60-80° C. and maintained for 10-15 minutes. The mass is stirred at 75-80° C., maintained for 25-35 minutes at 70-80° C., and stirred for 2 hours at 70-80° C. The solvent is distilled completely under vacuum at 70-80° C. for 35-40 minutes. 1-Methoxy-3,5-di-O-acetyl-D-ribofuranose (37.28 g), and dichloromethane (460 mL) are added to the residue and stirred for 5-10 minutes. The mass is cooled to 0-5° C. and TMS triflate (33.18 mL) is added and stirred for 10-15 minutes at 0-10° C. The mass is gradually heated to 25-30° C. and stirred for 1-1.5 hours. The mass is cooled to 10-20° C. The reaction is quenched by adding 3% sodium carbonate solution (460 mL) and the mixture is stirred for 5-10 minutes. The layers are separated at 18-25° C. The aqueous layer is extracted with dichloromethane (460 mL). The combined organic layer is cooled to 0-5° C. and stirred for 40-50 minutes, and then the mass is filtered through a Hyflow bed and washed with dichloromethane (460 mL). The filtrate is distilled under vacuum for 50-60 minutes at 35-45° C. Yield: 16.8 g (60.4%); Purity: 55.80% (β-anomer).

### Example 5

### 1-(3,5-di-O-acetyl-2-deoxy-D-ribofuranosyl)-5-azacytosine

5-Azacytosine (5.0 g) and acetonitrile (25 g) are charged into a round bottom flask at 25-35° C. and stirred for 5-10 minutes. HMDS (98%, 19.1 mL) and ammonium sulphate (0.24 g) are added and the mixture is heated to 60-80° C. The mixture is stirred at 60-80° C. for 2-3 hours and cooled to 60-70° C. The solvent is distilled at 60-70° C. under a 350-400 torr vacuum for 20-30 minutes and dichloromethane (115 mL) and 1-methoxy 3,5-di-O-acetyl-D-ribofuranose (12.4 g) are added to the residue and stirred for 10-15 minutes at 25-30° C. The mass is cooled to 5-20° C. and TMS triflate (98%, 8.73 mL) is added and stirred for 10-20 minutes at 10-15° C. The mass is gradually heated to 25-30° C. and stirred for 1-1.5 hours. The mass is cooled to 10-20° C. and the reaction is quenched by adding 3% sodium carbonate solution (115 mL), then the mixture is stirred for 5-10 minutes and the layers are separated at 18-25° C. The aqueous layer is extracted with dichloromethane (115 mL). The combined organic layer is washed with water (115 mL) and distilled at 35-45° C. for 35-45 minutes. Isopropyl alcohol (40 mL) is charged to the residue, stirred for 1.5-2 hours, cooled to 0-5° C., and stirred for 10-15 minutes. The mass is filtered and the solid is washed with isopropyl alcohol (10 mL). The solid is dried at 25-40° C. for 5-6 hours. Yield: 4.4 g (33.2%); Purity: 47.37% (β-anomer).

### Example 6

### Preparation of Decitabine

1-(3,5-di-O-acetyl-2-deoxy-D-ribofuranosyl)-5-azacytosine (5 g) and methanol (10 mL) are charged into a round bottom flask. The mixture is stirred at 25-30° C. for 5-10 minutes and cooled to 10-15° C. 10% Methanolic ammonia solution (2×13.62 g) is added and stirred for 5-10 minutes at 10-15° C. The temperature is raised to room temperature and the mass is stirred for 3-3.5 hours. The mass is cooled to about 10° C. and stirred for 10-15 minutes. The formed solid is filtered, washed with methanol (2.5 mL), and suction dried to obtain decitabine. Yield: 1.55 g (83%); Purity by HPLC: 89%.

### Example 7

### Preparation of Decitabine

Methanolic ammonia (10%, 32.7 g) is charged at 15-25° C. into a round bottom flask, stirred for 5-10 minutes, and cooled to 10-20° C. 1-(3,5-di-O-acetyl-2-deoxy-D-ribofuranosyl)-5-azacytosine (2 g) is added and the temperature is raised to 20-30° C. The mixture is stirred at 20-30° C. for 4 hours and the formed solid is filtered, washed with methanol (2 mL), and suction dried to obtain decitabine. Yield: 25.17%; Purity: 97.2%.

### Example 8

### Purification of Decitabine

Decitabine (2 g, HPLC purity: 93.55%) and dimethylsulphoxide (12 mL) are charged at 25-35° C. into a clean round bottom flask, stirred for 5-10 minutes and cooled to 0-10° C. The mass is filtered through a Hyflow bed and the filtrate is divided into two equal parts.

i) Ethyl acetate (10 mL) is charged into a round bottom flask at 25-35° C. and stirred for 5-10 minutes. The first part of the filtrate is added and stirred for 35-40 minutes. The formed solid is filtered, washed with ethyl acetate (4 mL) and suction dried at 25-35° C. Yield: 0.65 g; HPLC purity: 96.23%.

ii) The second part of the filtrate is charged into a round bottom flask, stirred for 5-10 minutes at 25-35° C., then 2-butanol (10 mL) is added and stirred for 35-40 minutes. The formed solid is filtered, washed with 2-butanol (4 mL) and suction dried. Yield: 0.75 g. HPLC; purity: 98.47%.

### Example 9

### Preparation of Decitabine

10% Methanolic ammonia (1907 g) is charged into a dry round bottom flask and stirred for 10-15 minutes at 10-15° C. 1-(3,5-di-O-acetyl-2-deoxy-D-ribofuranosyl)-5-azacytosine (100 g) is added slowly and stirred for 5-10 minutes. The temperature is raised to room temperature and maintained for 30-40 minutes at 25-30° C. The mixture is stirred at 25-30° C. for 8-9 hours and cooled to 0-15° C. for 1 hour. The mixture is stirred for 7-8 hours at 0-5° C. and filtered. The solid is washed with methanolic ammonia (100 g) and dried at 25-40° C. for 8 hours for obtain 25.0 g of decitabine. Purity by HPLC: 99.2%; Impurity A: 0.06%; Impurity B: not detected; Impurity C: not detected; Impurity D: not detected; Impurity E: not detected; Impurity F and Impurity G: not detected; ROI: 0.1% w/w.

Decitabine (20.0 g) and dimethylsulphoxide (150 mL) are charged into a round bottom flask and stirred for 30-40 minutes at 25-45° C. The solution is filtered three times through a Hyflow bed at 25-30° C., and then the bed is washed with dimethylsulphoxide (10 mL). The filtrate is charged into a round bottom flask and a mixture of ethyl acetate (360 mL) and methanol (360 mL) is added. The mixture is maintained at 25-35° C. for 3-4 hours. Ethyl acetate (360 mL) is added at 25-35° C. and stirred for 5-6 hours. The formed solid is filtered and washed with a methanol and ethyl acetate mixture (100 mL:100 mL) and dried at 50-60° C. for 6-7 hours to give 13.5 g of the title compound. Purity by HPLC: 99.79%; Impurity A: 0.05%; Impurity B: not detected; Impurity C: not detected; Impurity D: not detected; Impurity E: not detected; Impurity F and Impurity G; not detected; ROI: 0.04% w/w.

Residual solvents: methyl acetate: not detected; ethyl acetate: not detected; methanol; 304 ppm; dichloromethane: not detected; isopropyl alcohol: not detected; acetonitrile: not detected; N,N-dimethylformamide: not detected; dimethyl sulphoxide: 1330 ppm.

The XRD pattern, DSC curve, and TGA curve are substantially in accordance with FIGS. 1, 2, and 3, respectively.

### Example 10

### Preparation of Decitabine

9.8% Methanolic ammonia (1459.4 g) is charged into a dry round bottom flask and stirred for 5-10 minutes at 25-30° C. and cooled to 15-20° C. for 10-15 minutes. 1-(3,5-di-O-acetyl-2-deoxy-D-ribofuranosyl)-5-azacytosine (75 g) is added and stirred for 25-35 minutes at 25-35° C. The mixture is stirred at 25-35° C. for 7-8 hours, cooled to 0-10° C. and stirred at the same temperature for 6-7 hours. The solid is filtered, washed with a solution of methanolic ammonia at 0-5° C. and suction dried at 25-35° C. The material is dried at 25-40° C. for 2.5-3 hours, then dried at 30-35° C. for 4-5 hours. The solid and dimethylsulphoxide (135 mL) are placed into a round bottom flask at 25-35° C. and stirred for 5-10 minutes. The mass is filtered through a paper filter. The filtrate is charged into a round bottom flask and stirred for 5-10 minutes at 25-30° C. A methanol-ethyl acetate (1:1) mixture (656 mL) is added at 25-30° C. and stirred for 3.5-4 hours. Ethyl acetate (328 mL) is added and stirred for 1.5-2 hours at 25-30° C. The solid is filtered, washed with the methanol-ethyl acetate mixture (180 mL) and suction dried. The solid material is dried at 55-65° C. for 11-12 hours.

Purity by HPLC: 99.81% (β anomer).

The XRD pattern and TGA curve are substantially in accordance with FIGS. 4 and 5, respectively.

### Example 11

### Hygroscopicity of Decitabine

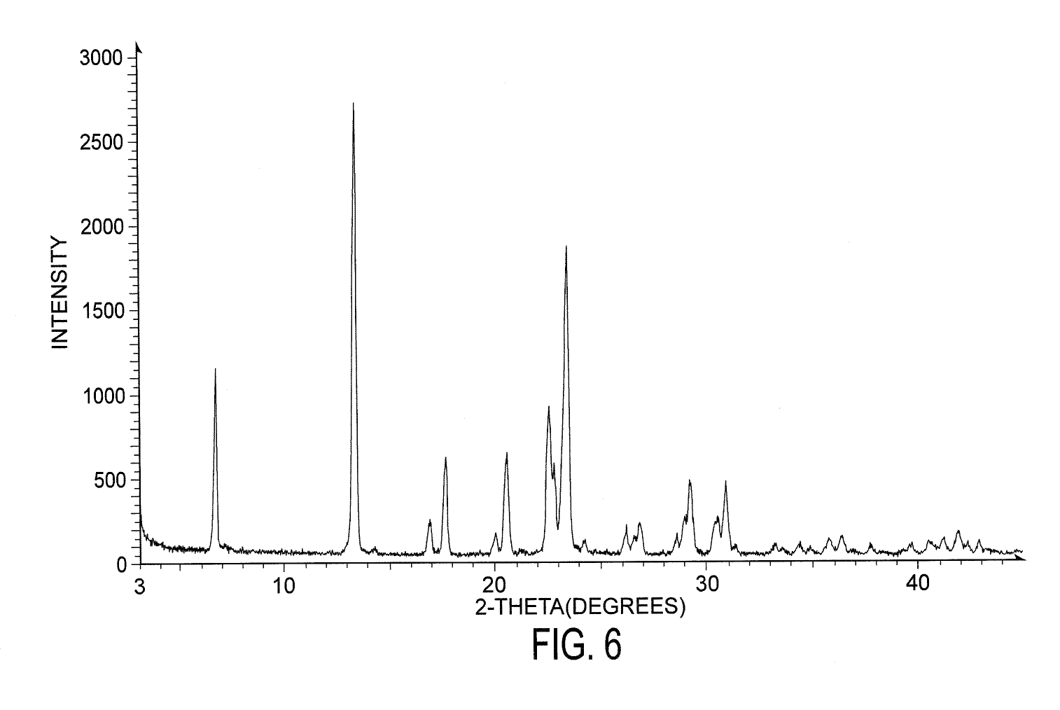

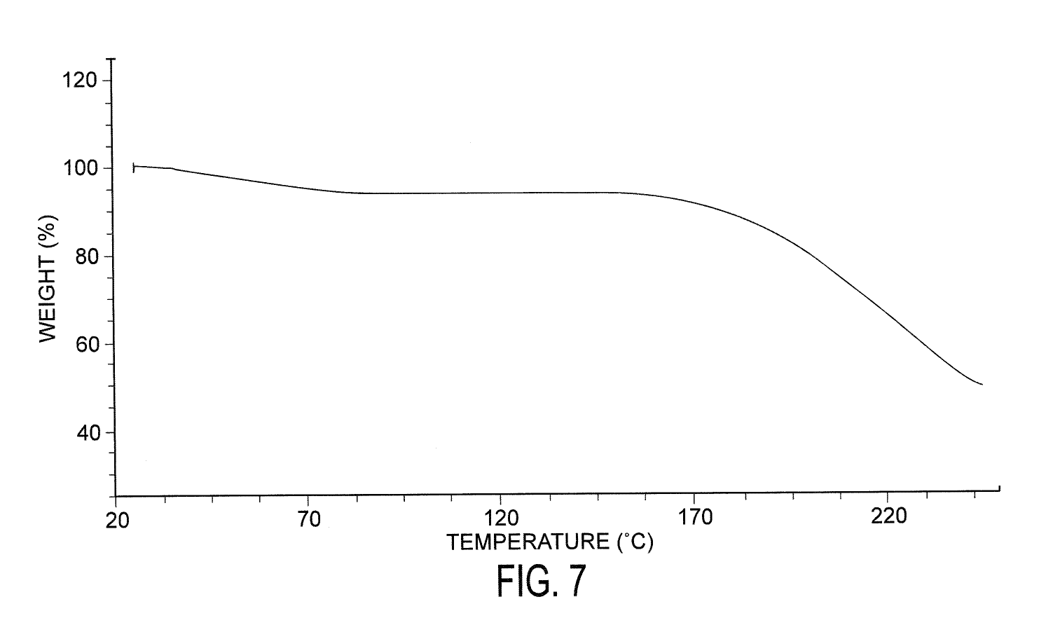

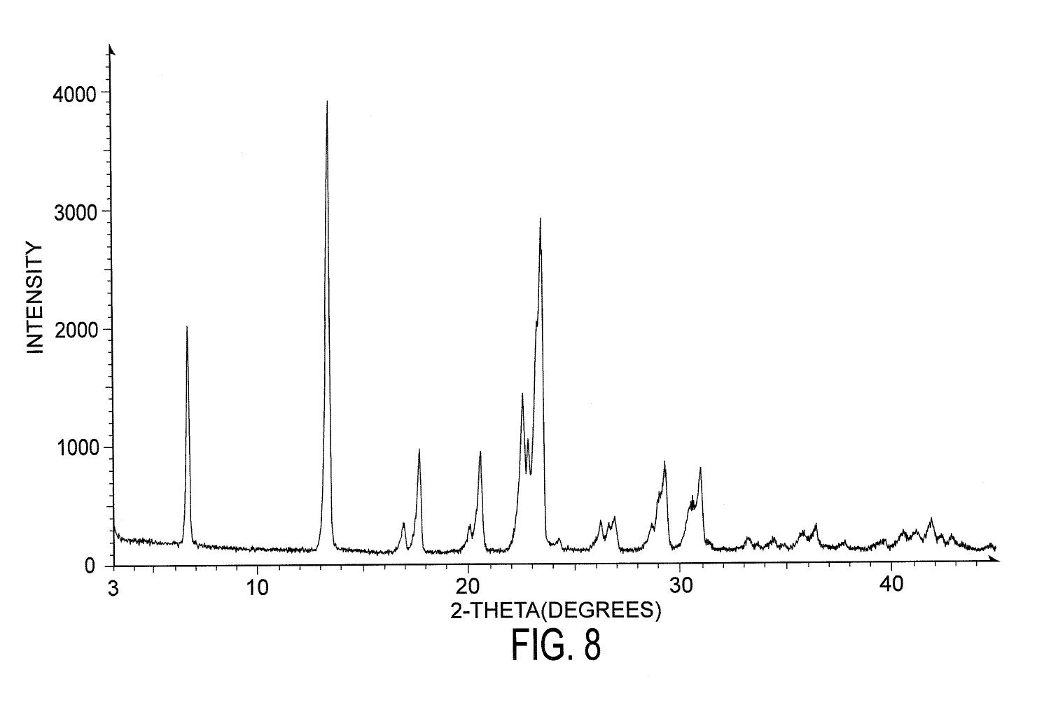

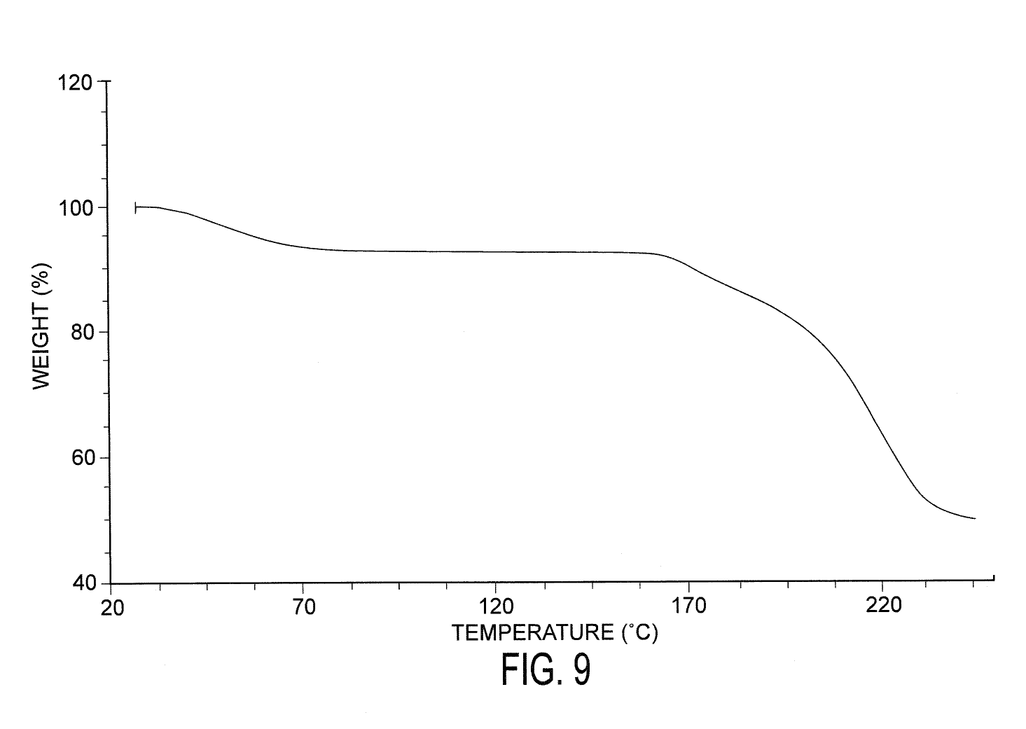

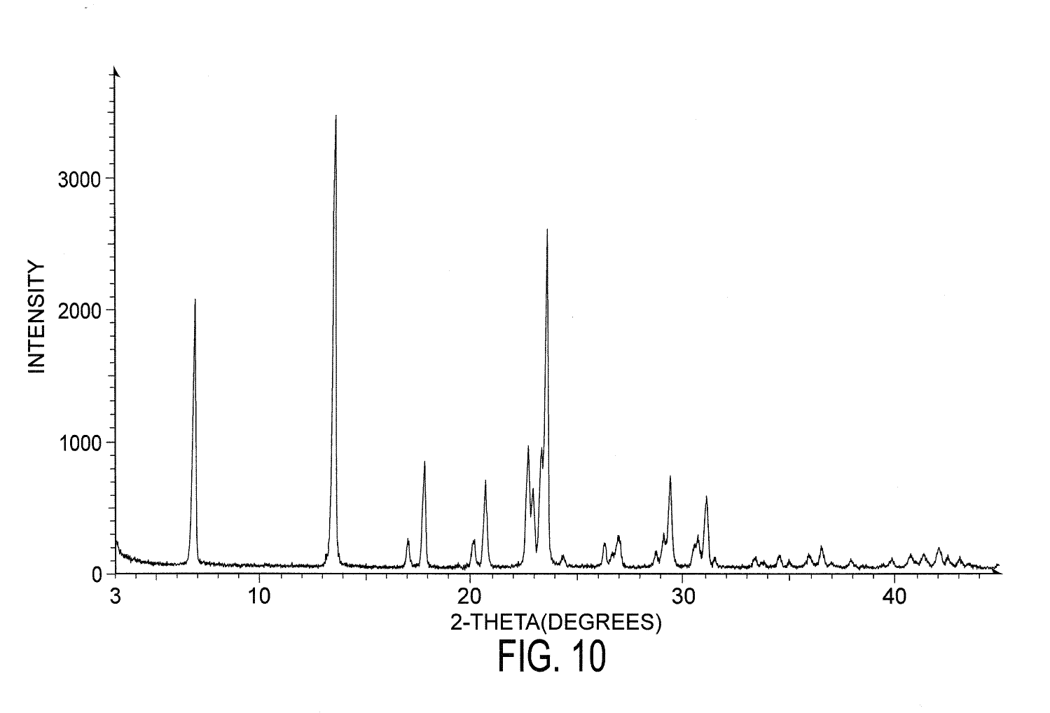

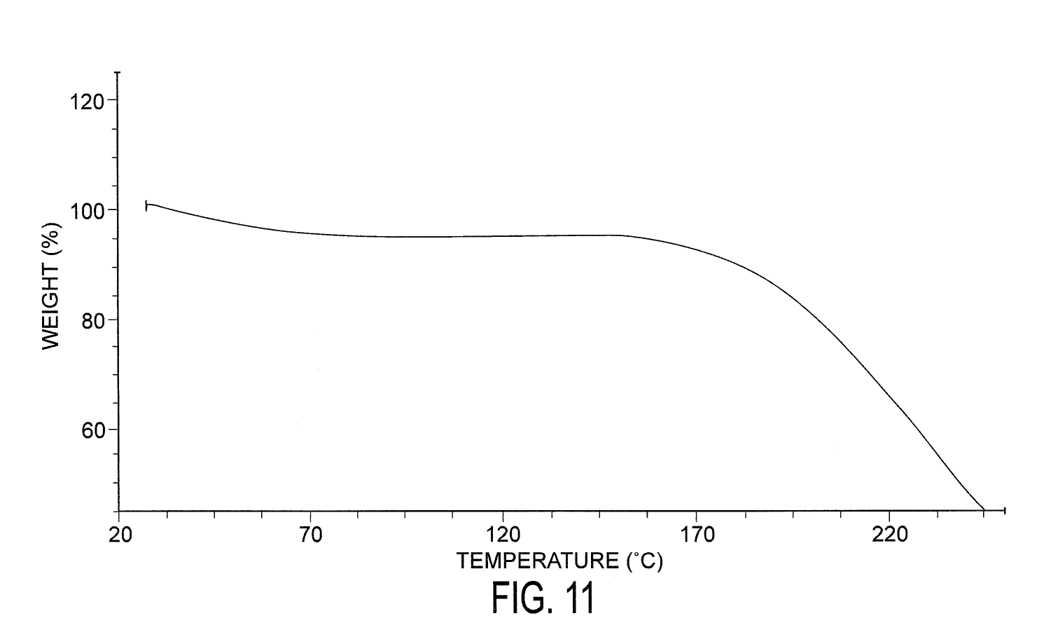

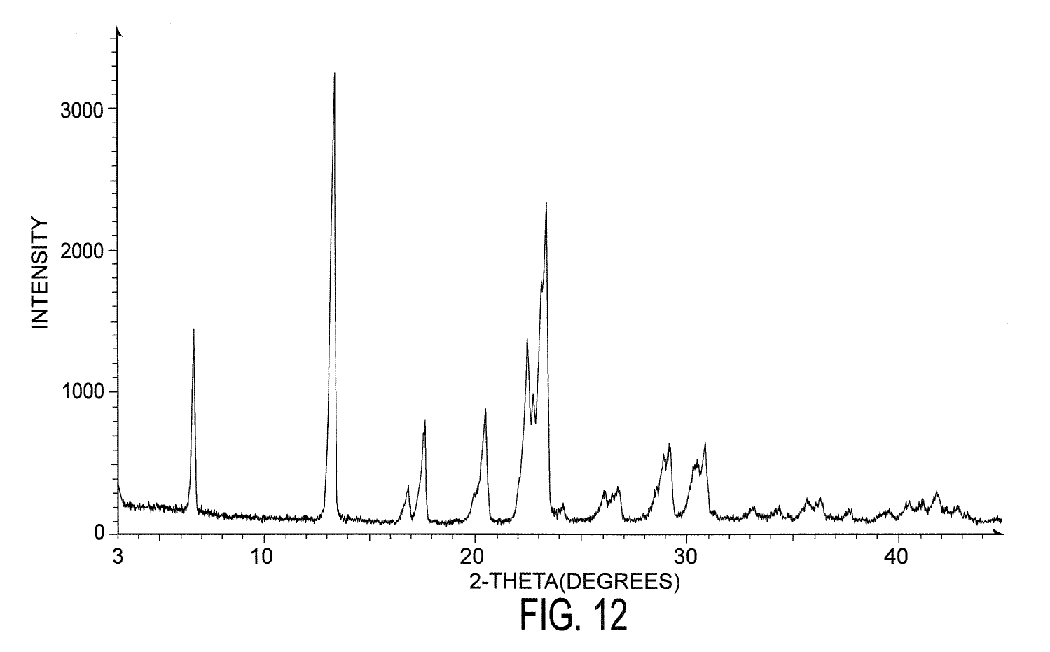

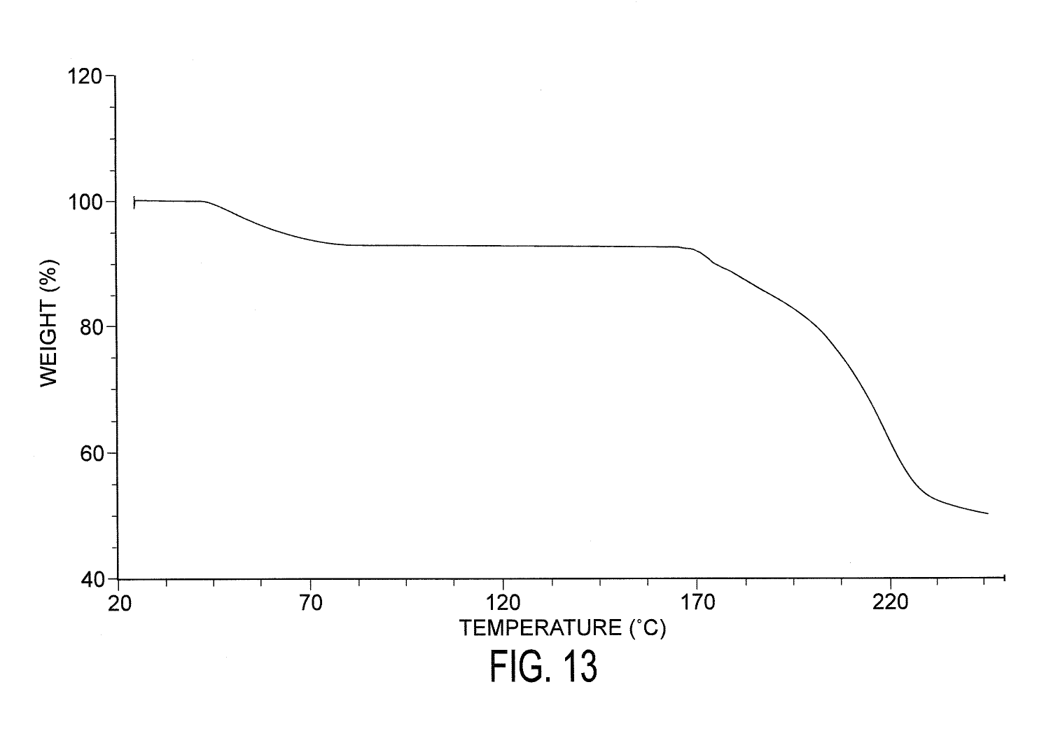

Decitabine obtained from Example 10 is tested for hygroscopic stability. Samples are analyzed as initially prepared, and at intervals after 1 day and 7 days of storage at room temperature; the results are tabulated below.

| Humidity | Time | Moisture (Wt. %) | PXRD Pattern | TGA Curve |
|---|---|---|---|---|
| 40-50% RH | Initial | 0.18 | FIG. 4 | FIG. 5 |
| | 1 day | 6.63 | FIG. 6 | FIG. 7 |
| | 7 days | 7.12 | FIG. 8 | FIG. 9 |
| 90% RH | Initial | 0.18 | FIG. 4 | FIG. 5 |
| | 1 day | 5.46 | FIG. 10 | FIG. 11 |
| | 7 days | 7.17 | FIG. 12 | FIG. 13 |

Decitabine obtained by processes of the present application, when exposed to the atmosphere, absorbs moisture and acquires a moisture content up to about 1 mole. This results in a change to a monohydrate form.

What is claimed:

1) A process comprising coupling the compound of formula (II): 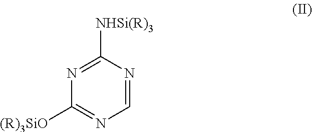
wherein each R independently is an optionally substituted C₁-C₆ alkyl group; with from about 0.9 molar equivalents to about 1.2 molar equivalents of a compound of formula (III): 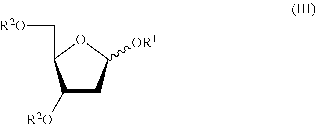
per molar equivalent of a compound of formula (II);
wherein R¹ represents an alkyl group having 1 to 6 carbon atoms and each R² independently is an optionally substituted acyl or aroyl group, in the presence of from about 1 molar equivalent to about 1.2 molar equivalents of a non-metallic Lewis acid, per molar equivalent of a compound of formula (II), and an organic solvent, to provide a beta-anomer enriched compound of formula (IV):

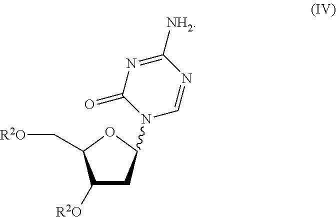

2) The process of claim 1 further comprising silylating 5-azacytosine in a nitrile solvent, to produce a compound of formula (II).

3) The process of claim 1 further comprising deprotecting the compound of formula (IV) to obtain decitabine of formula (I):

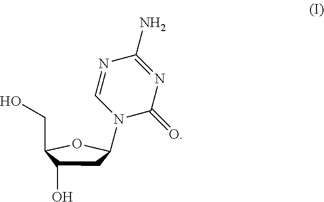

4) The process of claim 1, wherein the non-metallic Lewis acid comprises trimethylsilyl triflate or triisopropylsilyl triflate.

5) The process of claim 1, wherein the organic solvent comprises a halogenated hydrocarbon, an ester, a nitrile, or toluene.

6) The process of claim 2, wherein 5-azacytosine is silylated with a silylating agent in the presence of a catalyst.

7) The process of claim 3, wherein the deprotecting comprises reacting the compound of formula (IV) with a base and in the presence of an organic solvent.

8) The process of claim 7, wherein the base comprises an alkali metal hydroxide, a metal alkoxide, ammonia, or an organic base, and the organic solvent comprises a C₁-C₄ alcohol.

9) The process of claim 7, wherein the base comprises ammonia, and the product decitabine is substantially free of the mono acyl- or aroyl-protected decitabine of the formula (V): 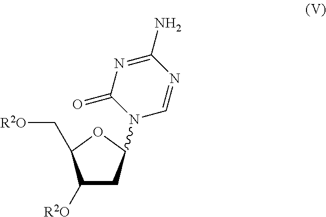
wherein each R² independently is hydrogen, an acyl, or an aroyl group, provided that both are not hydrogen, acyl, or aroyl.

10) A process comprising:
a) providing a solution or suspension of decitabine in an alcohol; and
b) forming a solid by cooling the solution the solution of step a).

11) The process of claim 10, wherein the alcohol comprises methanol, ethanol, or isopropanol.

12) The process of claim 10, wherein the solution of decitabine is provided in the presence of a base.

13) The process of claim 12, wherein the base comprises an alkali metal alkoxide, ammonia, or an organic base.

14) The process of claim 10, wherein the product obtained is a beta-anomer enriched product.

15) A processes for preparation of crystalline decitabine having characteristic XRPD peaks located at approximately 7.0, 13.0, 14.3, 18.5, 21.5, and 24.5±0.2° 2θ, or in the locations substantially as shown in FIG. 4, comprising:
a) providing a solution of decitabine in dimethyl sulphoxide; and
b) crystallizing a solid from the solution of step a).

16) The process of claim 15, wherein the crystallizing comprises combining the solution of step a) with an anti-solvent.

17) The process of claim 16, wherein the anti-solvent comprises an alcohol, an ester, or a mixture thereof.

18) The process of claim 17, wherein the anti-solvent comprises a methanol and ethyl acetate mixture.
